(19) United States
(12) Patent Application Publication
(10) Pub. No.: US 2010/0016774 A1
(43) Pub. Date: Jan. 21, 2010

(54) TREATMENT OF SOFT TISSUE INJURY IN CONJUNCTION WITH IMMOBILIZATION FOR PAIN TREATMENT

(76) Inventor: Edmund Wong, Honolulu, HI (US)

Correspondence Address: Howard Cohen, 1105 The Alameda, Berkeley, CA, 94707-2501, US

(21) Appl. No.: 11/632,803

(22) PCT Filed: Jul. 12, 2005
(86) PCT No.: PCT/US2005/024752

§ 371 Date: Jan. 16, 2007

Related U.S. Application Data
(60) Provisional application No. 60/589,263, filed on Jul. 19, 2004.
(60) Provisional application No. 60/589,264, filed on Jul. 19, 2004.

(51) Int. Cl. A61F 5/00

(52) U.S. Cl. 602/19

(57) ABSTRACT

A apparatus for treatment of acute or chronic pain comprising a pain relief treatment device applicable to a soft tissue injury at the periosteal-osseous boundary, and a device for immobilization of the area of soft tissue injury after application of the treatment device. Pain treatment may comprise injection of anti-inflammatory agent, including but not limited to steroids, and/or anesthetics; or low power laser irradiation, or cold treatment to the area of soft tissue injury. Immobilization devices for the cervical and lumbar vertebrae are provided for promoting healing of treated soft tissue injury sites. The invention includes a packaged set of materials comprising a pain relief treatment device, and an external supportive device for immobilizing the soft tissue injury at the site of treatment.

### CROSS-REFERENCE TO RELATED APPLICATIONS

This application claims the priority filing date of PCT application PCT/US2005/024752, filed Jul. 12, 2005, which is based on U.S. Provisional Applications 60/589,263 and 60/589,264, both filed Jul. 19, 2004.

### FEDERALLY SPONSORED RESEARCH

Not applicable.

### SEQUENCE LISTING, ETC ON CD

Not applicable.

### BACKGROUND OF THE INVENTION

1. Field of the Invention

This invention relates to the treatment of acute and chronic pain and, more particularly, to such pain caused by soft tissue injury at the periosteal-osseous interface.

2. Description of Related Art

Pain is an unpleasant sensation signaling that the body is damaged or threatened with an injury. Chronic headaches, muscle pain, joint pain, and the like are experienced by most individuals, and many persons have such chronic pain on a daily or weekly basis.

Of all the soft tissues of the body (muscles, tendons, ligaments and fatty connective tissue) the most common site of injury is at the periosteal-osseous junction. The weakest parts of the entire muscular-skeletal system are formed at these junctions where the long collagen fibers attach themselves to the cortical layer of the bone by very fine root-like structures. They embed themselves into 7-8 layers of lamellae of the bone. Sudden impact as found in an acceleration or deceleration motion will cause the fibers to tear at this junction of the hard and soft tissues. Such an injury, microscopic or macroscopic, may result in rupturing the tiny blood and lymphatic vessels, which are made up of a single layer of endothelial cells. The rupturing of these vessels and other connective tissue will release certain noxious chemicals such as kinins, bradykinins, potassium ions, serotonins, prostaglandins and others into the surrounding connective tissues, disrupting the entire chemical and osmotic balance. These chemicals stimulate the “C” fibers to cause the pain sensation. In this immediate vicinity of the injury, the noxious substances accumulation can lead to edema formation. Because of the pain, the sympathetic autonomic nervous system comes into play by causing the smooth muscles that surround the lymphatic vessels to constrict. This constriction has an adverse effect on the lymphatic vessels by preventing the pumping action that would otherwise evacuate the noxious chemicals which are causing the pain. Thus the pain persists.

Administration by injection of a mixture of anti-inflammatories and local anesthetics without a vasoconstrictor at the precise site(s) of the soft tissue injury(ies) will block the pain stimulus to the pain centers in the brain, thus giving temporary pain relief. Likewise, infrared laser treatment at these exact injury sites has been described in U.S. Pat. No. 5,640,978, issued Jun. 24, 1997. In addition, chilling through application of ice or cold water or cold objects for about 30 minutes can produce similar results. This period of pain relief, which may last for a few minutes to many hours, allows the lymphatic vessels to evacuate the noxious chemicals. In a short time, the ruptured blood vessels will seal by way of the coagulation of the blood plasma. Although the pain may be reduced or disappear within 30 second to a few minutes, it does not mean that the pain will not return. If the injury is microscopic, only a small amount of the noxious chemicals is present. However, if it is macroscopic, there can be major edema formation, with intense pain, and it may take a few minutes or more to evacuate these chemicals and obtain pain relief. Any stress placed on the tendenous or ligamentous attachment may abort the healing process by reinjuring the fragile tissues resulting in the release of noxious chemicals causing pain. To prevent reinjury, immobilization following treatment with injection, laser, or cold treatment is paramount.

Frequently, medical treatment consists of drugs such as analgesics or muscle relaxants. These substances are systemic, and can have adverse side effects such as kidney toxicity, liver inflammation, gastrointestinal symptoms, and the like. Alternative treatments consist of chiropractic manipulations, acupuncture, physical therapy, stress relief regimens, and the like. These approaches to treatment have had limited success, as they are focused on the symptoms rather than the cause.

Pain is often felt at the actual site of the tissue injury; however, in some instances pain felt in some areas of the body may not accurately represent where the injury is, because pain can be “referred” to another area. The term “referred pain” has been used to describe pain that is experienced at a locus removed from the cause or lacking an identified cause.

With regard to referred pain, research conducted 50 and 60 years ago indicated that referred pain could be emulated in test subjects by injecting hypertonic saline solution into the tips of the spinous processes of the vertebrae and causing temporary inflammations of the periosteum from the base of the skull to the sacrum. Referred pain was produced at various sites remote from the injection points, and was virtually indistinguishable from the sensations described by chronic pain sufferers. Moreover, it was clear that induced inflammation of specific sites along the spinous processes resulted in pain sensation at corresponding specific sites throughout the body far removed from the cause, and that the pain sensation could also be induced in the form of headaches similar to migraine headaches. This research also established that the pain referral mechanism did not involve mere neural transmission. However, this promising early work apparently was not followed, and did not result in effective treatment modalities. However, this early research provided a great insight to the understanding of pain to the inventor.

The present invention provides an approach for treating pain that differs from previous methods by relying on immobilization of the area of soft tissue injury following treatment of the soft tissue injury through either administration of anti-inflammatory and/or local anesthetic agents without a vasoconstrictor agent, laser treatment, or cold treatment. The invention further provides immobilization devices for the cervical and lumbar portions of the spine to prevent movement of those areas to allow the injured, treated tissues to heal. It also provides a system for treating pain, by providing treatment agents combined with appropriate immobilization devices in a prepackaged kit for medical practitioners.

### BRIEF SUMMARY OF THE INVENTION

The present invention comprises, in one aspect, a method for treatment of acute or chronic pain comprising treatment of the soft tissue injury(ies) that are the cause of the pain, in conjunction with immobilization of the area of soft tissue injury giving rise to the pain, so that the treated injuries are given an opportunity to heal. The pain to be treated includes, but is not limited to, pain generated from stimulation of A-fiber and C-fiber nerve endings (neurogenic pain), as well as the neuromuscular pain resulting from the phenomenon of the withdrawal reflex. Note that C-fibers are highly concentrated in the periosteum tissue, and are often stimulated by soft tissue injuries at the periosteal-osseous boundary.

The method is particularly useful in treating acute and chronic headaches and migraine headaches, as well as pain of the upper and lower back, neck, shoulders, hip, knee, arm, leg, and pain associated with carpal tunnel syndrome. The method is also useful for treatment of pain resulting from musculoskeletal and musculotendon injuries. A significant aspect of the invention is that the method treats the causative factor of the acute and chronic or referred pain, so that the method achieves pain reduction or relief and ultimate cure of the pain through healing of the causative injuries. Yet another significant aspect of the invention is the immobilization of the area of soft tissue injury following treatment. Yet another significant aspect of the invention is the utilization of ergonomic and posturing protocols to prevent reinjury.

It has been found in most instances that the source of referred pain such as headaches, backaches (upper and lower back), neck pain, shoulder and knee pain and carpal tunnel syndrome involves microscopic and macroscopic tears in the periosteal-osseous junctions of the vertebrae, the scapula, and the base of the skull. Additionally, pain may result from musculoskeletal and musculotendon junction tears at different locations of the body. Such injuries release histamines, kinins, bradykinin, prostaglandins, proteolytic enzymes, serotonin, and other substances. These substances not only cause C-fiber pains but are responsible for numerous localized autonomic reactions, such as muscle spasm, ischemia, local inflammation, edema, as well as generalized reactions such as increased blood pressure, photophobia, nausea, blurred vision, copious mucous flow of the nose and sinuses, and the like. The muscle spasms are responsible for transmitting the pain sensation to other portions of the body.

The site(s) of soft tissue injury(ies) may be treated by injection of an anti-inflammatory substance mixed with an anesthetic agent directly to the lesion site(s). Note that the anesthetic agent should be devoid of any vasoconstrictor substance, which would thwart the function of the lymphatic system. Alternatively, a low power infrared laser may be use to irradiate the sites. As a further alternative, cold treatment of the sites may be carried out. In all three of these alternative treatments, the effect is to relieve the pain by allowing the lymphatic system to expel the noxious substances from the injured site that are the cause of the edema and pain sensation, whether localized or referred pain. Some or all of these treatment modes may be applied by the patient, or by an individual who is not medically trained.

It is an essential element of the present invention that, following treatment of the lesion site, the injured site is immobilized immediately with the specialized devices of the invention. Immobilization is necessary to enable the normal healing process to take place by preventing movement that would otherwise re-injure the treated sites before they are fully healed. Immobilization of the site of injury is in contrast to the usual treatment of exercise prescribed following treatment of injuries. Such immobilization may be accomplished using a variety of different types of external supportive devices, i.e., braces. The types of braces to be used will depend on the site to be immobilized, i.e., neck, back, or knee brace, to name a few.

A significant aspect of the invention is the provision of immobilization devices that are designed to protect sites of soft tissue injury after they are treated in accordance with the invention. One device comprises a brace adapted to circumscribe the lower abdomen with an elastic band assembly that supports a fluid bag. The fluid bag is disposed to impinge on the lumbar spine to establish a proper curvature to the lumbar vertebrae and resist movement that could reinjure soft tissue lesions in the lumbar area. Another device comprises a brace designed to support the cervical vertebrae. It includes an adhesive patch placed on the forehead, and a tension band extending rearwardly therefrom along the midline of the scalp. At the distal end of the tension band, a pair of tension lines diverge laterally to extend to a pair of adhesive patches placed on upper rear portions of the shoulders in generally symmetrical relationship. The tension applied by the lines to the band causes the head to be held erect, so that the cervical vertebrae are maintained in an erect, more vertical column configuration. This arrangement, unlike the prior art cervical collar, enables the patient to turn the head, but prevent slumping or slouching posture that may reinjure soft tissue injuries in the cervical area.

A further aspect of the invention is the provision of a packaged set of materials, such as a kit, comprised of a pain relief treatment device and/or substances together with the appropriate cervical or lumbar brace, as described above. In the instance of injection treatment, the anti-inflammatory and anesthetic agents may be provided separately or in a pre-mixed combination. The kit may preferably include the syringe used for injection, so that a user may be supplied in one package with all the materials necessary to carry out the treatment. Alternatively, the kit may include a device for providing low power laser light, or a device for applying cold treatment to the soft tissue injury site(s). The kit may also include a manual for instructing a user on the application of the pain relief treatment and the proper use of the immobilization device.

### BRIEF DESCRIPTION OF THE DRAWINGS

FIGS. 1 and 2 are anatomical renderings of the musculature of the head, neck, and back, taken from Gray's Anatomy, Fifteenth Edition, Gramercy Books, 1977.

FIGS. 3 and 4 are side and rear elevations, respectively, of a human skull, depicting some of the soft tissue treatment sites in accordance with the method of the invention.

FIGS. 5 and 6 are posterior and side elevations, respectively, of the cervical human vertebrae, depicting some of the treatment sites in accordance with the method of the invention.

FIG. 7 is a posterior view of the upper human skeleton, showing treatment points in accordance with the method of the invention.

FIG. 8 is a posterior elevation of the sub-occipital triangle attachment of muscles, showing treatment points in accordance with the method of the invention.

FIGS. 9 and 10 are side and rear elevations, respectively, depicting a cervical neck brace in accordance with the invention.

FIG. 11 is a perspective view of the lumbar brace in accordance with the invention.

FIG. 12 is a front elevation of a patient wearing the lumbar brace depicted in FIG. 11.

FIGS. 13 and 14 are schematic side elevations of the human body, showing portions of the thorax, abdomen, and hip, before and after application of the lumbar brace of the invention.

FIG. 15 is a perspective view of a treatment kit in accordance with the invention.

### DETAILED DESCRIPTION OF THE INVENTION

The present invention, in one aspect, generally comprises a system for treatment of acute and chronic pain comprising treatment of soft tissue injury(ies) at the periosteal-osseous boundary which evacuates the edema containing the noxious chemicals that cause the pain, in conjunction with immobilization of the area of injury following treatment. The method of the present invention may also be used in conjunction with the use of ergonomics and posturing protocols to prevent reinjury. The method is particularly useful in treating referred pain such as that associated with acute and chronic headaches and migraine headaches, as well pain of the upper and lower back, neck and shoulders, hip, arm, leg, and pain associated with carpal tunnel syndrome. Additionally, the method of the invention may be used to treat musculoskeletal injuries and musculotendon junction injuries.

The invention is based on the principle that the source of referred pain such as headaches, backaches (upper and lower back), neck pain, shoulder pain and other areas involves microscopic and macroscopic tears in the periosteal-osseous junctions. Upon injury, the tissues liberate histamines, kinins, bradykinins, prostaglandin, proteolytic enzymes, serotonin, and other substances. The resulting muscle spasms caused by these substances create pain sensations at locations which may be far removed from the insulting injury. Moreover, the site(s) of the periosteal-osseous lesion usually can be correlated directly with the locus of the pain sensation(s).

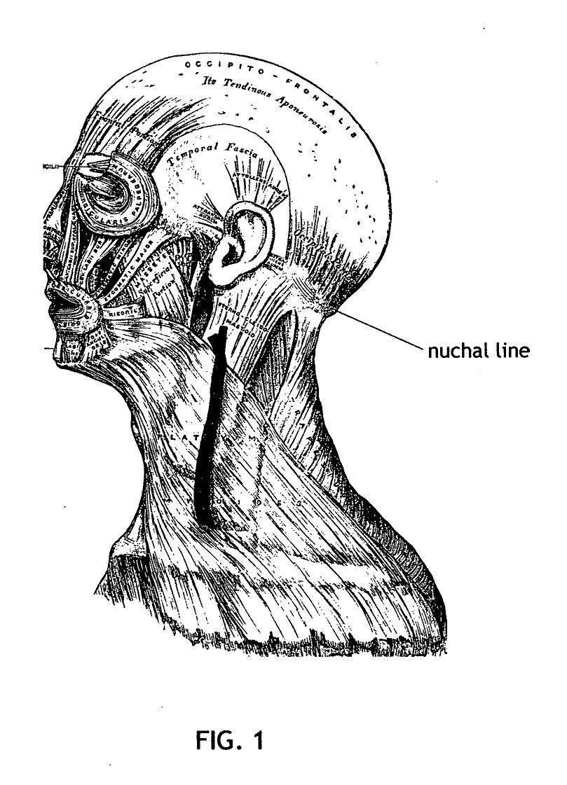

For example, the withdrawal reflex is the basis of neuromuscular dysfunction causing chronic headaches, migraine headaches, and the like. Noxious chemical stimuli from soft tissue injuries at the nuchal line of the skull cause the occipitalis muscles to spasm and withdraw, activating the golgi end organs and muscle spindles of the occipitalis, and the anterior, posterior, and superior auricularis muscles (see FIG. 1). Tension placed on the aponeurosis of the scalp is extended to the frontalis and orbicularis oculi muscles. The involvement of the auricularis muscles accounts for headache pain sensation at the sides of the head, and the involvement of the orbicularis oculi muscles is the cause of pain sensations about the eyes. However, these pain sensations are due to soft tissue injuries along the nuchal line adjacent to the occipital protuberance, far removed from the loci of pain sensation.

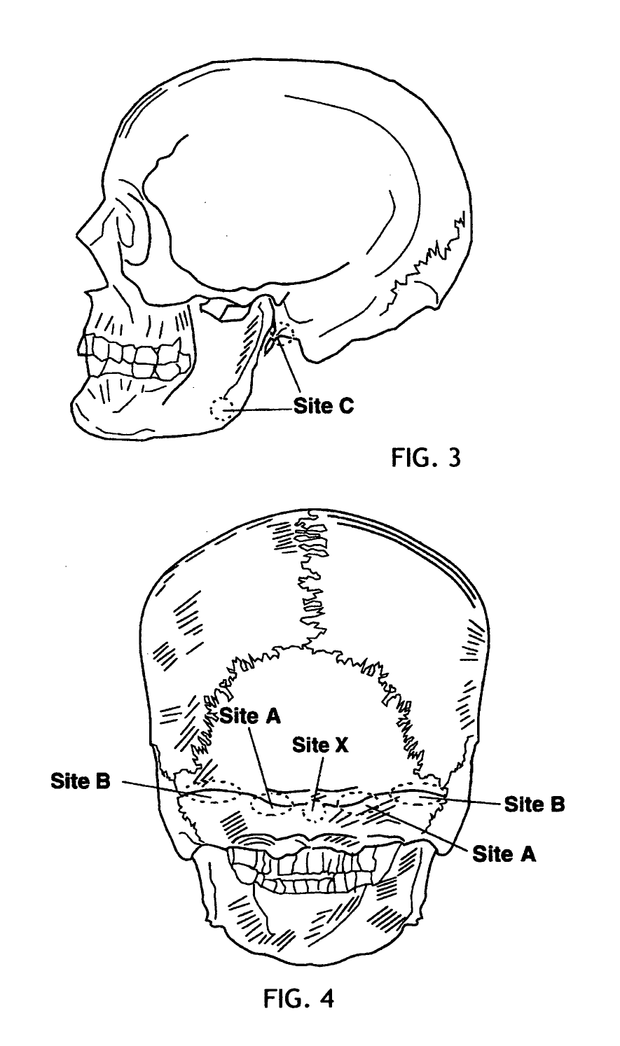

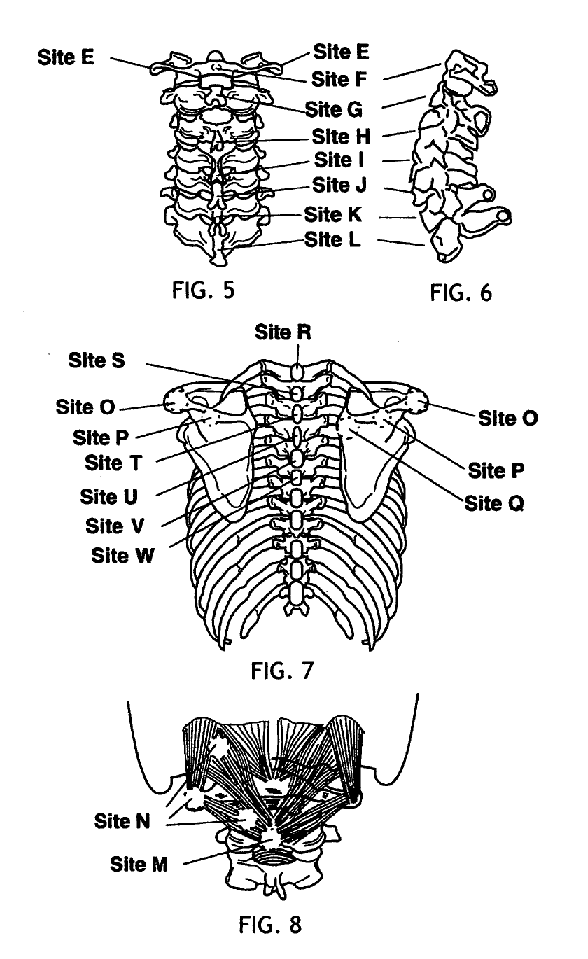

To treat the cause of pain at different locations in the body, treatment is administered to the sites of the insulting injury, including but not limited to those indicated in the following charts (see FIGS. 3-8):

TABLE I
HEADACHES

| Pain Location | Treatment Site |
|---|---|
| Occipital | Superior nuchal line of occipital bone and |
| | occipital protuberance - Sites A, X, B |
| Frontal headache, | Nuchal line from occipital protuberance to |
| intra-ocular pains- | medial ⅓ of superior nuchal line - Sites A, X |
| lateral aspect | |
| of nasal bone | |
| Temporal, Parietal | Lateral ⅓ of superior nuchal line, especially |
| and intra-ocular | above mastoid process - Site B |
| pain | |
| Mastication pain | Tip of the transverse process of C1; tips of |
| | styloid process and angle of the mandible |
| | (SMD) over TMJ - Site C |

TABLE II
NECK PAIN

| Pain Location | Treatment Site |
|---|---|
| Anterior Cervical | Styloid process and angle of mandible (SMD) - |
| | Site C |
| Lateral Cervical | Lateral and inferior surface of mastoid process; tip |
| | of transverse process of C1; attachment of |
| | trapezius at nuchal line; tip of spinous process of |
| | C2 - Sites D, E, F, G, and M |

TABLE III
POSTERIOR CERVICAL PAIN

| Pain Location | Treatment Site |
|---|---|
| Upper cervical- | Medial ⅓ of nuchal line and occipital |
| superficial pain | protuberance - Site A |
| Upper cervical- | Inferior surface of nuchal line from deep pain |
| deep pain | ligamentum nuchae to mastoid process - Site N |
| Lower cervical | Tips of spinous processes of C2 and C4-C7, medial |
| | and superior angle of scapula and spine and |
| | acromion process - Sites G, I, J, K, L, O, P, Q. |

TABLE IV
PARASTHETIC WEAKNESS PAINS OF EXTREMITIES

| Pain Location | Treatment Site |
|---|---|
| Upper arm | Tips of spinous process of C4 and C5 - Sites C, I, |
| and forearm | and J. |
| Hands and | Tips of spinous process of C5, C6, C7 and T1 - |
| fingers | Sites C, J, K, L, and R. |
| Lower back, | Tips of spinous process of C6, C7, and feet T1 |
| legs and feet | and/or T2, T3, T4, T5, and T6 - Sites C, K, L, R, S, T, |
| | U, V, W. |

The initial step of relieving the pain at the soft tissue injury site comprises, in one mode, the administration by injection of anti-inflammatory and/or anesthetic agents to the area to be treated. Anti-inflammatory agents include those compounds capable of reducing the inflammatory response associated with pain. In a preferred embodiment of the invention, the anti-inflammatory agent is a steroid compound. Such steroidal compounds include, but are not limited to, prednisone, prednisolone, methylprednisone, betamethasone, dexamethasone, and cortisone. In addition or in place of anti-inflammatory agents, anesthetic agents may also be utilized to treat pain. Such agents include, for example, procaine (Novocain), lidocaine, prilocaine, and mepiscaine. These agents should be free of any vasoconstrictor substance, which would defeat the function of the lymphatic system. The anti-inflammatory agents reduce inflammation and help restore lymphatic circulation, so that the noxious chemicals released by the soft tissue injury may be removed by the lymphatic system. In addition, the anesthetic agents reduce or eliminate the pain sensation, which releases the muscle contractions caused by the pain sensation and interrupts the referred pain process. Release of muscle spasms also promotes blood circulation which accelerates healing of the soft tissue injury.

Determination of doses to be administered can easily be established by one of ordinary skill in the art. In general, the dose to administered is that dose sufficient to reduce the sensation of pain. The anti-inflammatory and/or anesthetic agents may be administered using a variety of different methods, including, for example, injection of such agents into the area of tissue injury.

In a further mode of the invention, the treatment sites described herein may be irradiated using a low power laser. This technique is described in U.S. Pat. No. 5,640,978, issued Jun. 24, 1997 to the present inventor, and is incorporated herein by reference. The laser produces coherent light in the near- to mid-infrared range so that the beam penetrates the overlying tissue and reaches the lesion. In a preferred embodiment the wavelength of the laser is in the range of 800-900 nm. Lasers having wavelengths of 805-830 nm have been used with great efficacy. The power level employed ranges from 10-150 milliwatts, with an upper limit of 500 milliwatts, and the treatment time ranges from a few seconds to an upper limit of 75 minutes. Beam energy density is in the range of 200 mw per cm². Treatment time, power level, and reiteration is correlated with the extent of the soft tissue lesions and the depth of the overlying muscle and tissue. Treatment may be terminated based on a timed dose of laser energy; however, the patient often spontaneously notices pain remission or relief, and treatment may be stopped at that time. Determination of laser energy doses can be easily determined by one of ordinary skill in the art. Lasers having appropriate wavelength and power characteristics are commercially available.

In another mode of the invention, the treatment sites described herein may be treated by cold treatment, involving the use of ice, cold compress or similar cold object for 30 minutes at least. If the soft tissue injury is in the lumbar spine area, the lumbar brace of the invention may also be used for the cold treatment by chilling the fluid bladder before applying the brace to the patient. In addition, a plurality of fluid bladders may be provided so that a chilled bladder may replace a bladder in the brace after it has been warmed by absorbing heat from the lumber spine area.

In all the modes noted herein, the soft tissue injury(ies) are treated to relieve the pain sensation and release the muscle withdrawal and tension that accompanies the pain. The lymphatic system is then able to evacuate the noxious substances that have been generated by the injury and have caused the localized edema and pain sensations. Thereafter immobilization is carried out to protect the treated site(s) from movement and promote healing.

Pains in the neck can be treated to stimulate healing of the soft tissue injuries that are responsible for the pain sensations. Many forms of lower back pain are also treatable in accordance with the method of the present invention. The most common sites for soft tissue injuries are at the inferior and superior surfaces of the spinous and transverse processes in acceleration and deceleration injuries. With these tears in the periosteum, hemorrhage can be expected, accompanied by release of histamines, bradykinins, serotonin, potassium ions, prostaglandins and the like. These substances become the noxious stimuli that cause the superficial and deeper paravertebral muscles to contract. The sustained contractions result in muscular spasms that compress the vertebral disks. In the presence of subclinical herniated or protruding disks, muscle spasms cause the disk to protrude posteriorly, resulting in lower back or leg pain. If the disk protrudes laterally, the patient may complain of chest pain brought about by contraction and spasm of the intercostals muscles that extend anteriorly. This same mechanism is responsible for some parasthetic weakness of the arms, hands, and fingers. In the case of lower back pain and parasthetic weakness, treatment is administered to the site of the soft tissue injury(ies) to promote healing and removal of the causative factor. The treatment parameters are as described previously.

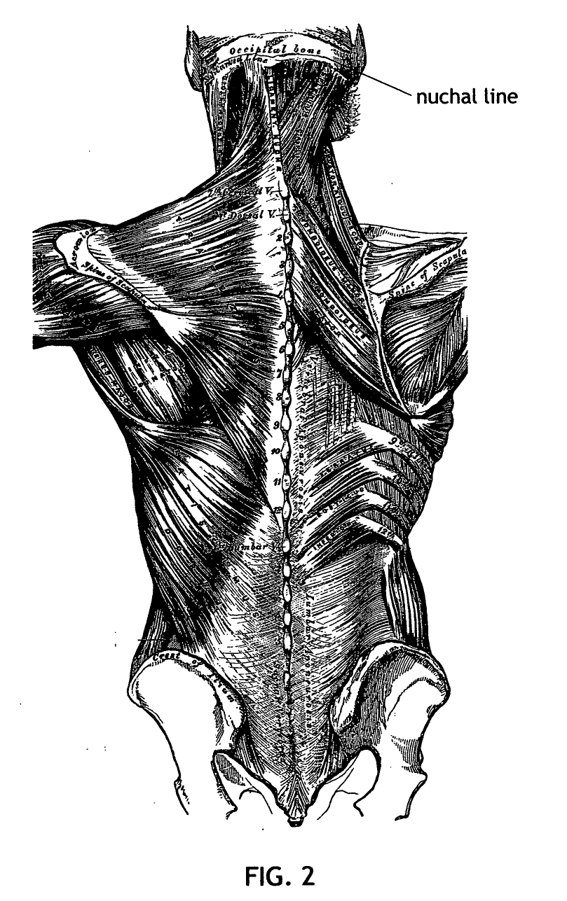

The present invention further relates to the treatment of patients suffering from “carpal tunnel syndrome” (CTS). The majority of CTS patients have faulty postures with their head and neck stooped forward and their shoulders rounded. The abnormal posture is the consequence of the slow evolution of ongoing soft tissue injuries of the cervical spine and shoulders. On examination, they also have tenderness at the tips of spinous processes C5-T1, medial angle, superior border and the spine of the scapula. See FIG. 2.

Subjects suspected of having CTS should have a complete examination of the cervical spine area and shoulders to identify sites of injury. Once the injured site is identified, such as by palpation and detection of edema indicative of soft tissue injury, the site is treated by administration of an anti-inflammatory and/or anesthetic agent, or laser light, or cold treatment. Following treatment, the areas of injury are isolated and immobilized by taping or bracing until healing has taken place. Faulty posture of the cervical spine and shoulders can be corrected by bracing and strengthened by appropriate, timely, and carefully regulated exercise, after treatment and healing has been completed.

For all of the treatment sites noted above for headache, neck, and extremities, it is necessary to palpate the recommended treatment sites to detect any edema at the sites. Often the patient will notice tenderness, pain, or sensitivity upon touch or pressure applied to the treatment sites, although these sensations were not the presenting symptoms. Such a reaction is a good indication that these treatment sites are indeed loci of soft tissue injuries, and should be treated with an anti-inflammatory and anesthetic agent devoid of any vasoconstrictor, or laser energy, or cold treatment.

Remedying the injury requires time to clear the injured environment of pain chemicals, and time for the damaged tissue to heal. In the same way that a skin laceration cannot heal if it is subject to repeated movement and strain, or that a broken bone cannot mend without being immobilized after being properly set, bracing and immobilizing damaged soft tissue are equally important to implement until the soft tissue injury has sufficient time to heal. Thus, healing the injury necessitates immobilization of the damaged soft tissue and prevention of any movements that may reinjure the soft tissue.

Thus, once the soft tissue treatment is complete, an external supportive device is used to immobilize the area of the soft tissue injury undergoing healing, so that a cure is effected. Such external supportive devices include, for example, back, neck, knee or wrist braces. The area of soft tissue injury is immobilized for a sufficient amount of time so that healing occurs. Physical therapy and exercises are appropriate only when the injury has sufficiently healed; if performed too early or too vigorously, the damage can be exacerbated, extended, painful and more difficult to repair. Additionally, ergonomics and posturing protocols may be utilized to prevent reinjury.

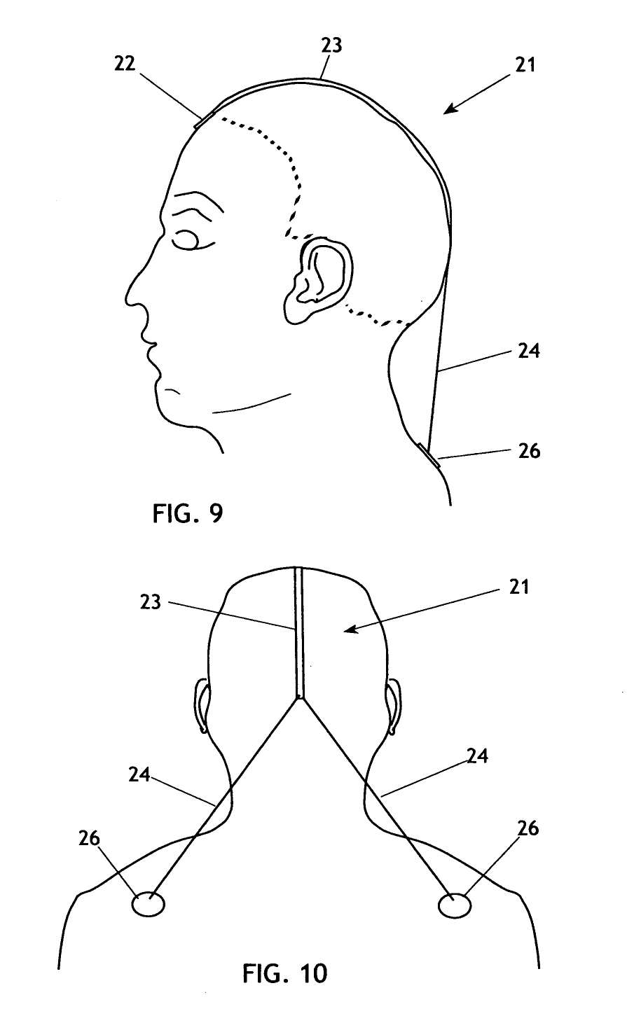

With regard to FIGS. 9 and 10, the invention provides a head support device 21 that is designed in particular to carry out the methodology of the invention. The device 21 is used following treatment of soft tissue injuries in the cervical area (sites E-L), as well as along the nuchal line and the occipital protuberance (sites A, B, and X). The device 21 is contrived to maintain an erect posture and natural curvature of the cervical vertebrae while enabling sufficient rotation of the head to carry out normal functions, unlike cervical collar braces that prevent rotation and interfere with eating, talking, and the like.

Head support device 21 includes an adhesive patch 22 that is placed on a medial portion of the forehead of the patient, the patch being preferably as transparent and unobtrusive as possible. Joined to the patch 22 is a tension web 23 which extends rearwardly and medially along the scalp of the patient to a distal end near the occipital protuberance of the skull. A pair of tension lines 24 are joined to the distal end of the web 23 and extend downwardly and laterally outwardly at symmetrical, oblique angles. The distal ends of the tension lines 24 are joined to respective adhesive patches 26, which are secured to upper outer portions of the shoulders of the patient in generally symmetrical lateral relationship. Note that the hair of the patient may be parted to receive the tension web 23 and then combed thereover. Likewise, the tension lines 24 may extend through an open collar shirt or blouse in an unobtrusive manner.

The tension lines 24 are adjusted in length so that the patient's head is held in an erect posture, with the cervical vertebrae disposed in its natural curved configuration. The tension applied from the lines 24 through the web 23 to the forehead of the patient elevates the forehead and prevents the head from slumping forward into a position that might reinjure the treated soft tissue injuries of the cervical spine and nuchal line. However, the chin of the patient is completely free and unfettered, so that eating, telephone use, shaving and washing, etc. may be carried out without hindrance. Likewise, the assembly 21 permits the patient to rotate the head laterally without obstruction, except for extreme rotations that might otherwise affect the healing of the treated injuries. Further, there is free air circulation about the neck of the patient to prevent excessive perspiration in warm conditions. Note that the head may be tilted rearwardly without interference, but any forward drooping motion will be resisted by the tension applied through components 23 and 24.

The device 21 may be easily installed and removed by anyone, such as a medical practitioner or by the patient. Once the tension lines are properly dimensioned, the patient may remove the device by removing the adhesive patches (as for bathing and showering) and reinstalled with minimal effort.

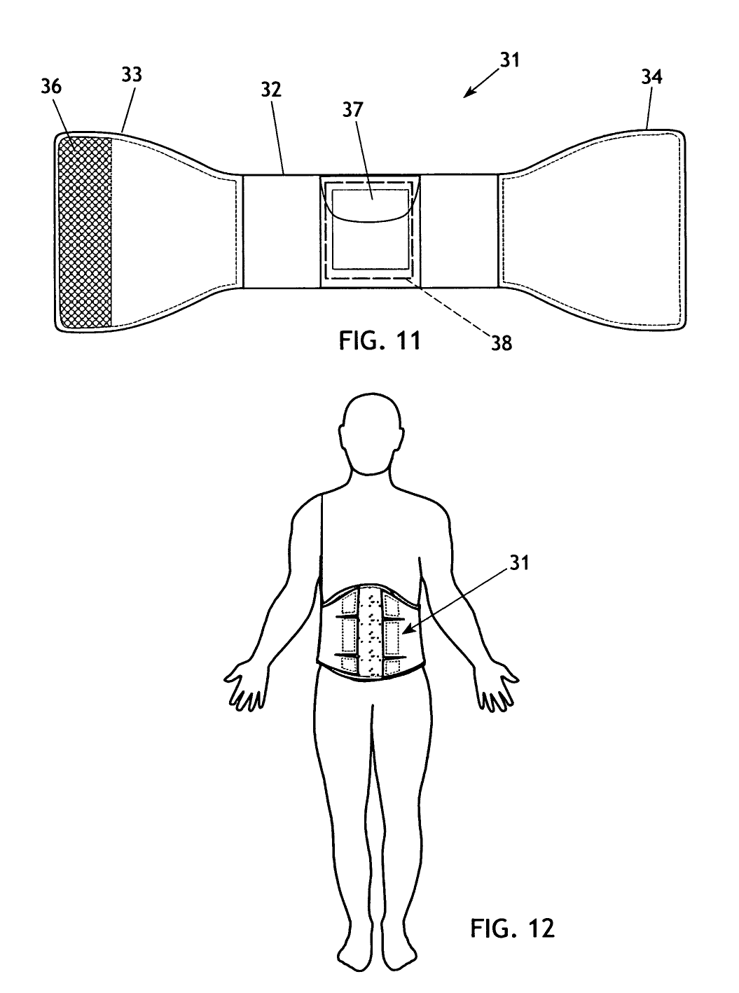

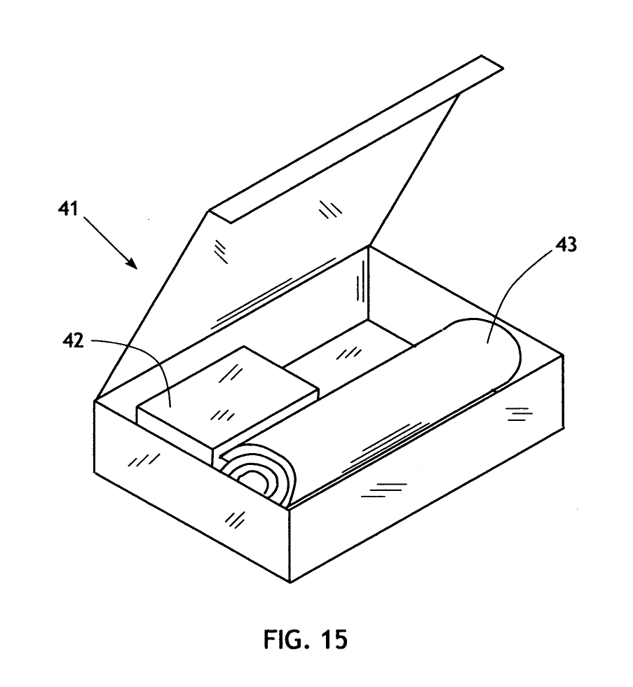

With regard to FIGS. 11-14, the invention also provides an immobilization device 31 for use in immobilizing the lumbar vertebrae region following injection treatment of the lumbar vertebrae. The device 31 includes a medial band 32, a laterally elongated rectangular assembly that is comprised of stretchable, elastic material, and a pair of front panels 33 and 34 joined to the laterally opposed ends of the medial band 32. The panels 33 and 34, which are also formed of an elastic material or assembly, are flared in width as they extend from the medial band 32. Panel 33 is provided with a loop fastener portion 36 on the front surface (as viewed in FIG. 11), and panel 34 is provided with a hook fastener portion on the back surface (not shown).

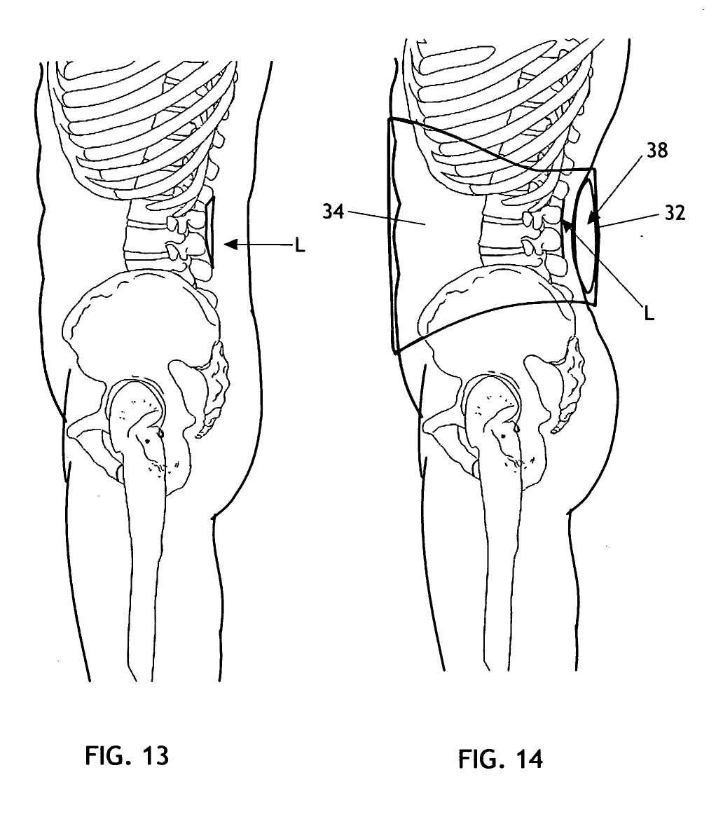

Disposed in the middle of the medial band 32 is a pocket 37, which is dimensioned to receive and retain a fluid bladder 38. The fluid bladder is generally rectangular, and is filled with a medically acceptable gel material that is may be more viscous than water but not necessarily form-retaining. The fluid bladder 38 is disposed to impinge on the lumbar portion of the spinal column and provide support and immobilization therefore. With regard to FIG. 13, the lumbar portion L of the vertebral column is a locus of chronic and referred pain caused the soft tissue injuries at the periosteal-osseous boundary, leading to pain that may be felt in the lower back, leg, and hip. After the soft tissue injury site(s) are treated with anti-inflammatory and/or anesthetic agents, or laser energy, or cold treatment, the device 31 is applied to the patient. The bladder 38 is arranged to impinge directly on the concave curvature (the lumbar curve) of the lumbar spine, and the medial band is extended laterally about the lower back of the patient. The front panels 33 and 34 are wrapped about the front of the lower abdomen, in overlapping fashion, and placed under resilient tension. The hook and loop fasteners are then engaged to secure the device 31 about the lower abdomen. Note that the wide front panels serve to distribute the resilient compressive force broadly over the front of the abdomen, whereas the narrower medial band acts to focus the resilient compressive force on the fluid bladder 38 and its impingement on the lumbar spine. The fluid bladder adapts its contours to the lumbar curve on which it impinges to provide maximum comfort, and the constant pressure that it exerts in a uniform manner on the lumber spine supports the vertebrae and urges them to align properly. That is, the natural lumbar curve is encouraged and supported, which places the muscles supporting the lumbar spine in a condition of minimal muscular contraction and balance. Muscle spasm is eliminated so that the injury sites may be allowed to heal. It also immobilizes the soft tissue injury sites that have been treated. Thus the support of the device also tends to correct faulty posture, which may be the cause of the pain symptoms undergoing treatment. Thus further tissue damage is prevented, and the injuries are stabilized and healing is promoted.

The level of resilient compressive force is adjustable by virtue of the wide hook and loop portions, which enable an adjustable degree of overlap and tension in the assembly. The fluid bladder 38 is removable from the pocket 37, and may be heated or refrigerated to supply the appropriate thermal effects. Likewise, two or more bladders 38 may be provided and exchanged, so that one may be in place in the device 31 while the other(s) are undergoing heating or cooling for subsequent use. Thus the device 31 may be used to carry out the cold treatment portion of the invention, as well as the immobilization portion.

The present invention further relates to a packaged set or kit of materials designated for carrying out the treatment steps of the invention. With regard to FIG. 15, the kit 41 may comprise (i) device for soft tissue injury treatment in a container 42; and, (ii) an external supportive device 43 for immobilization of the area of soft tissue injury. The container 42 may house the an anti-inflammatory and/or anesthetic agent; pharmaceutical agent(s) and, where permissible by regulation or custom, a syringe for injecting them. Alternatively, the container 42 may house a device for providing a low power laser beam. The device 43 may comprise the immobilizing devices 21 and/or 31, or other such devices known in the prior art. In the instance of cold treatment of the soft tissue injury, the container 42 may house one or more fluid bladders 38 to facilitate chilling one bladder while another one is in use in the brace 31. The packaged set of materials of the invention is designed for use by subjects in need of pain treatment and is designed for easy self-treatment, or for use by individuals without medical training. In this regard, the packaged materials of the invention may further comprise an instruction manual for use of the anti-inflammatory and/anesthetic agents, the laser device, and the external supportive device.

The foregoing description of the preferred embodiments of the invention has been presented for purposes of illustration and description. It is not intended to be exhaustive or to limit the invention to the precise form disclosed, and many modifications and variations are possible in light of the above teaching without deviating from the spirit and the scope of the invention. The embodiment described is selected to best explain the principles of the invention and its practical application to thereby enable others skilled in the art to best utilize the invention in various embodiments and with various modifications as suited to the particular purpose contemplated. It is intended that the scope of the invention be defined by the claims appended hereto.

1. A system for treatment of pain in a human patient, including:
a device for providing immediate pain relief for at least one site of a periosteal-osseous soft tissue injury that is causing pain;
an external immobilizing support device for immobilizing the area surrounding the site of soft tissue injury following utilization of the device for immediate pain relief, whereby said at least one soft tissue injury is limited in movement and permitted to heal.

2. The system for treatment of pain of claim 1, wherein said device for providing immediate pain relief includes means for administering a pain relief substance selected from a group consisting of anti-inflammatory substances and anesthetic substances.

3. The system for treatment of pain of claim 1, wherein said device for providing pain relief includes a low power laser for irradiating the soft tissue injury site.

4. The system for treatment of pain of claim 1, wherein said device for providing pain relief includes means for administering cold treatment to the soft tissue injury site.

5. The system for treatment of pain of claims 2 to 4, wherein said immobilizing device is adapted to immobilize the cervical vertebrae for treatment of soft tissue injuries in the cervical area.

6. The system for treatment of pain of claim 5, wherein said external support device includes a first adhesive patch applied to the medial forehead of the patient, and tension means secured to said first adhesive patch and extending rearwardly therefrom to elevate the head and maintain the cervical vertebrae in a generally vertical column.

7. The system for treatment of pain of claim 6, wherein said tension means includes a tension band having a proximal end secured to said first adhesive patch, said tension band extending rearwardly from said first adhesive patch and disposed medially along the scalp of the patient.

8. The system for treatment of pain of claim 7, wherein said tension means further includes a pair of tension lines having like first ends secured to a distal end of said tension band; a pair of a second adhesive patches, each secured to an upper shoulder portion of the patient, said tension lines having like second ends each secured to one of said second adhesive patches, said tension lines being under tension to exert tensile force through said tension band to said first adhesive patch and maintain elevation of the head of the patient.

9. The system for treatment of pain of claim 2 to 4, wherein said external support device is adapted to immobilize the lumbar vertebrae for treatment of soft tissue injuries in the lumbar area.

10. The system for treatment of pain of claim 9, wherein said external support device includes an elastic assembly adapted to circumscribe the abdomen of the patient, and a fluid bladder supported by said elastic assembly in impingement on the lumbar vertebrae.

11. The system for treatment of pain of claim 10, wherein said elastic assembly includes a medial band adapted to impinge on the lower back of the patient, and a pair of front panels, each secured to opposed ends of said medial band and adapted to impinge on the front abdomen of the patient, and means for securing said front panels in adjustable fashion at the front of the abdomen.

12. The system for treatment of pain of claim 11, wherein said front panels are substantially wider than said medial band, whereby resilient compressive force exerted by said elastic assembly is widely distributed on the front abdomen of the patient and focused on said fluid bladder at the back of the patient.

13. The system for treatment of pain of claim 12, wherein said medial band includes means for removably securing said fluid bladder in position to impinge on the lumbar vertebrae of the patient.

14. The system for treatment of pain of claim 11, further including a plurality of said fluid bladders that are interchangeably supported by said medial band.

15. The system for treatment of pain of claim 14, wherein said fluid bladders are filled with a gel substance capable of being heated to apply heat treatment to the lumbar vertebrae, or chilled to apply cold treatment to the lumbar vertebrae.

16. The system for treatment of pain of claim 12, wherein said resilient compressive force urges said fluid bladder to impinge on and support the lumbar vertebrae in a generally vertical column.

17. The system for treatment of pain of claim 2, wherein said means for administering a pain relief substance includes an injector.

18. The system for treatment of pain of claim 3, wherein said low power laser comprises an infrared laser having a power output in the range of 10-500 mW.

19. The system of pain relief of any one of claims 17 further including a set of instructions for use of the injector and external support device.

20. A kit for treatment of pain in a human patient, including:
a pain relief device for treating at least one site of a periosteal-osseous soft tissue injury that is causing pain;
an external support device for immobilizing the area surrounding the site of soft tissue injury following the pain relief treatment, whereby said at least one soft tissue injury is limited in movement and permitted to heal.

21. The kit of claim 20 further comprising a set of instructions for use of the pain relief device.

22. The kit of claims 19 or 20, wherein said pain relief device includes an injector and a pain relief substance selected from a group consisting of anti-inflammatory substances and anaesthetic substances.

23. The kit of claims 19 or 20, wherein said pain relief device includes a low power laser.

24. The kit of claims 19 or 20, wherein said pain relief device includes a cold treatment device.

25-47. (canceled)

48. A system for use in the prevention of further injury comprising:
a pain relief treatment for administration to at least one site of a periosteal-osseous soft tissue injury that is causing pain;
an external support device for immobilizing the area surrounding the site of soft tissue injury following administration of the pain relief treatment.

49-69. (canceled)

70. A device for supporting and immobilizing the lumbar vertebrae, including:
an elastic assembly adapted to circumscribe the abdomen of a patient and having a medial band adapted to impinge on the lower back of the patient, and means for securing said elastic assembly in adjustable fashion at the front of the abdomen;
means for removably securing a fluid bladder in position to impinge on the lumbar vertebrae of the patient with resilient compressive force to urge said fluid bladder to impinge on and support the lumbar vertebrae in a generally naturally curved vertical column.

71. A device for supporting and immobilizing the cervical vertebrae, including:
a first adhesive patch adapted to be applied to the medial forehead of a patient, and a tension band secured to said first adhesive patch and extending rearwardly therefrom;
a pair of tension lines having like first ends secured to a distal end of said tension band; a pair of a second adhesive patches, each secured to an upper shoulder portion of the patient, said tension lines having like second ends each secured to one of said second adhesive patches, said tension lines being under tension to exert tensile force through said tension band to said first adhesive patch and maintain elevation of the head of the patient and maintain the cervical vertebrae in a generally vertical column.
